(19) United States
(12) Patent Application Publication
(10) Pub. No.: US 2004/0068182 A1
(43) Pub. Date: Apr. 8, 2004

(54) Digitally reconstruced portal image and radiation therapy workflow incorporating the same

(76) Inventor: Satrajit Chandra Misra, Fremont, CA (US)

Correspondence Address: Siemens Corporation, Attn: Elsa Keller, Legal Administrator, Intellectual Property Department, 186 Wood Avenue South, Iselin, NJ, 08830, US

(21) Appl. No.: 10/246,160

(22) Filed: Sep. 18, 2002

(51) Int. Cl. A61B 5/05

(52) U.S. Cl. 600/427

(57) ABSTRACT

Digitally reconstructed portal imaging systems and methods and radiation therapy workflow systems and methods incorporating the same are described. Among these systems and methods are systems and methods for planning a radiation therapy treatment, verifying radiation therapy treatment, verifying patient position in a radiation therapy treatment, and computing a portal image.

### TECHNICAL FIELD

This invention relates to radiation therapy systems and methods.

### BACKGROUND

Radiation therapy involves delivering an ionization dose (curative/palliative) of radiation to a tumor, while minimizing the dose delivered to surrounding healthy tissues and adjacent healthy organs. Therapeutic radiation doses typically are supplied by a charged particle accelerator that is configured to generate a high-energy electron beam. The electron beam may be applied directly to one or more therapy sites on a patient, or it may be used to generate a photon (e.g., X-ray) beam, which is applied to the patient. With a multi-leaf collimator, the geometry of the radiation beam at the therapy site may be controlled by multiple leaves that are positioned to block selected portions of the radiation beam. The multiple leaves may be programmed to contain the radiation beam within the boundaries of the therapy site and, thereby, prevent healthy tissues and organs located beyond the boundaries of the therapy site from being exposed to the radiation beam. A tumor may be treated by multiple beams that are delivered with varying doses and geometries from several different angles. Intensity modulated radiation therapy (IMRT) is a technique in the treatment of tumors that involves the delivery of many small, concentrated radiation beams, each of which may deliver a different dose. In this approach, an IMRT planning and optimization process automatically determines beam parameters (e.g., beam geometry, beam directions, beam weights, etc.) based upon stated clinical objectives.

In general, after a tumor has been discovered in a patient, the radiation oncologist works with a number of radiation therapy specialists to develop a radiation therapy treatment plan. The radiation oncologist monitors the delivery of the radiation therapy treatment to the patient, and verifies that the tumor has been treated properly. Initially, the radiation oncologist selects a set of parameters that define the clinical objectives for treating the patient. The radiation oncologist then sends these parameters to a dosimetrist who then develops one or more proposed radiation therapy treatment plans that satisfy the clinical objectives. A typical radiation therapy plan calls for the delivery of a series of radiation treatment fractions to the patient over the course of several days or weeks. Each treatment fraction consists of a sequence of radiation segments with a prescribed cumulative dose intensity profile. The doctor sends a radiation therapy plan selected from the set received from the dosimetrist to a physicist who verifies the plan and determines whether the plan can be implemented on a selected treatment system through a QA (Quality Assurance) process. After the plan has been verified by the physicist, the doctor sends the final radiation therapy plan to a radiation therapy technician who will irradiate the patient in accordance with the final plan. During and after treatment, the doctor reviews portal images that were captured during treatment to verify that the radiation was delivered to the tumor accurately and at the correct dosage level.

### SUMMARY

The invention features the use of digitally reconstructed portal imaging systems and methods and radiation therapy workflow systems and methods incorporating the same.

In one aspect, the invention features a method of planning a radiation therapy treatment. In accordance with this inventive method, proposed treatment plan portal images are computed based upon a proposed treatment plan. Quality assurance portal images are acquired by irradiating a target volume having a known composition and geometry with radiation in accordance with a quality assurance treatment plan corresponding to the proposed treatment plan. The proposed treatment plan is evaluated based on a comparison of the proposed treatment plan portal images and the quality assurance portal images.

In another aspect, the invention features a method of verifying radiation therapy treatment. In accordance with this inventive method, a composite portal image IMRT map is computed based on an intensity modulated radiation therapy (IMRT) treatment plan and computed tomography (CT) data for a patient. Radiation delivered to the patient is evaluated based at least in part on the composite portal image IMRT map and a treatment portal image acquired during delivery of a target dose of radiation to a target volume of the patient in accordance with the IMRT treatment plan.

In another aspect, the invention features a method of verifying patient position in a radiation therapy treatment. In accordance with this inventive method, a reference portal image is computed based on a prescribed beam and computed tomography (CT) data for a patient (step a). The treatment portal image for the beam is acquired (step b). Patient position is adjusted based on a comparison of the reference portal image with the acquired portal image (step c). Steps (a)-(c) are repeated until the reference portal image and the acquired portal image match substantially.

In another aspect, the invention features a method of computing the dose delivered to the target during treatment. In accordance with this inventive method, a projection is computed for each beam of an IMRT field of a treatment plan based at least in part on transmission impact of patient volumes through which beams of the IMRT field respectively pass. The computed beam projections are summed to obtain a reconstructed composite portal image IMRT map. The dose and the variations in dose delivered to the target are computed by comparing the reconstructed composite portal image to the treatment composite portal image.

Other features and advantages of the invention will become apparent from the following description, including the drawings and the claims.

### DESCRIPTION OF DRAWINGS

FIG. 1 is a diagrammatic view of a radiation therapy workflow.

FIG. 2 is a block diagram of a simulation engine that is operable to compute digitally reconstructed portal images based on computed tomography data and a treatment plan.

FIG. 3 is a diagrammatic perspective view of a radiation source irradiating a target volume and a portal imaging device capturing radiation passing through the target volume.

FIG. 4 is a flow diagram of a method of planning a radiation therapy treatment.

FIG. 5 is a flow diagram of a method of verifying radiation therapy treatment.

### DETAILED DESCRIPTION

In the following description, like reference numbers are used to identify like elements. Furthermore, the drawings are intended to illustrate major features of exemplary embodiments in a diagrammatic manner. The drawings are not intended to depict every feature of actual embodiments nor relative dimensions of the depicted elements, and are not drawn to scale.

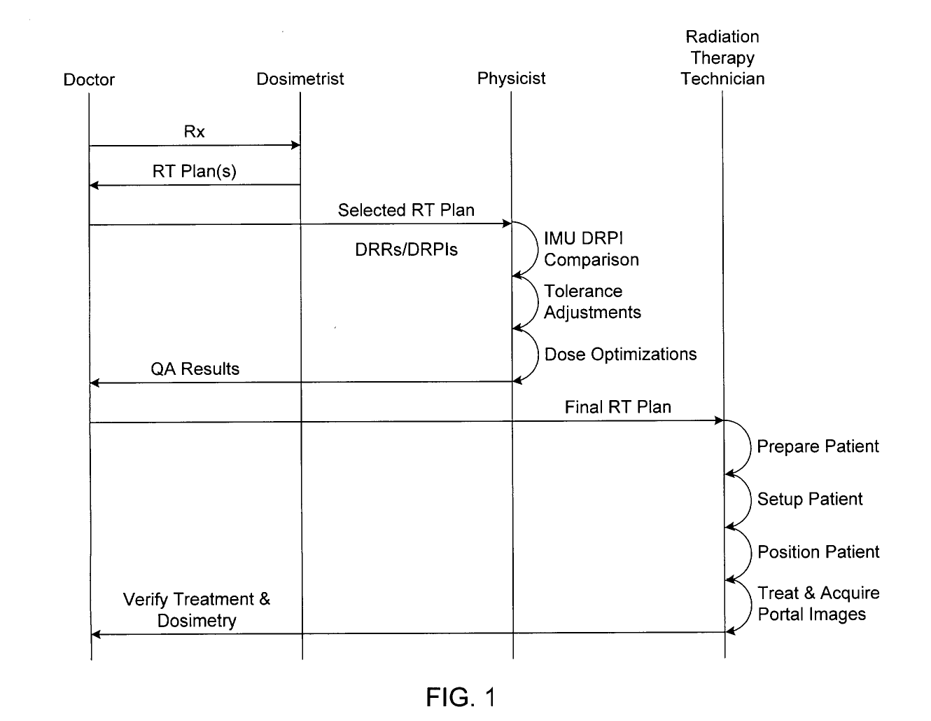

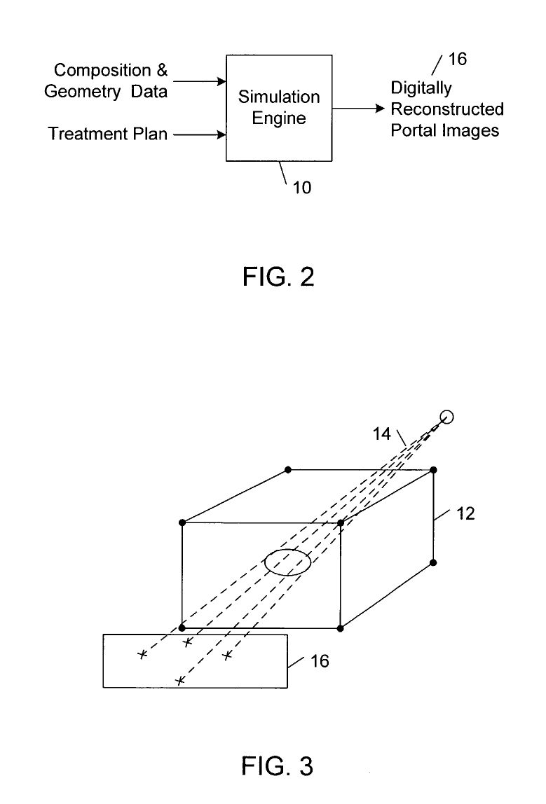

Referring to FIGS. 1, 2, 3, 4, 5 and 6, and initially to FIGS. 1 and 2, in some embodiments, a radiation workflow (or process) may be implemented as follows. After a patient has been diagnosed with cancer and external radiation treatment has been selected as the treatment method, a doctor (or oncologist) will determine a set of parameters (Rx) that define the clinical objectives to be met by the radiation treatment. In general, these parameters may include the location of the target tumor, the optimal intensity profile and the optimal curative dose for treating the tumor, and the locations of sensitive regions of the patient to spare from excessive radiation exposure. The clinical objective parameters are passed to a dosimetrist who generates one or more radiation therapy plans (RT Plan(s)) that satisfy the clinical objectives.

Each radiation treatment plan may involve the delivery of several treatment fractions to the therapy site over the course of several days. The goal of the treatment plan is to deliver a high curative dose to the tumor, while minimizing the dose received by normal tissues. The cumulative dose that may be delivered to a patient at any given time typically is limited by the radiation dose tolerance of critical healthy structures near the therapy site. The process of delivering an optimal treatment that conforms to the shape of the tumor typically involves modulating the intensity of the radiation beam across the beam dimension (i.e., perpendicular to the beam axis). Modulation of the beam intensity may be achieved by dividing the beam into a sequence of radiation segments each having a uniform intensity profile and a different beam shape, each shape being defined by the programmed position of the collimator leaves of a radiation therapy system.

After the doctor has selected a radiation treatment plan (Selected RT Plan) from the set of plans received from the dosimetrist, the doctor passes the selected plan to a physicist, who will perform multiple quality assurance tests on the selected plan to verify that it will satisfy the clinical objectives and can be implemented properly by the radiation therapy system that will be used to deliver the radiation therapy treatment.

Referring to FIGS. 2 and 3, in some embodiments, in addition to the details of the selected radiation treatment plan, the doctor may transmit to the physicist one or more images, which may be used by the physicist to conduct the quality assurance tests. These images may include both conventional digitally reconstructed radiograph (DRR) images, which correspond to simulations of images obtained from low-energy imaging beams, and digitally reconstructed portal (DRPI) images, which correspond to simulations of portal images obtained from high-energy (e.g., MeV) radiation treatment beams. Because a DRPI is generated based on treatment-level radiation, it corresponds more accurately to the treatment that a patient receives than a DRR image. A digitally reconstructed portal image may be generated by inputting into a simulation engine 10 data relating to the selected treatment plan (specified, e.g., in the form of a DICOM RT plan) and data relating to the composition and geometry of a target volume 12. At the planning stage, the composition and geometry data typically relates to the actual patient computed tomography (CT) data, whereas at the QA stage, the composition and geometry data typically relates to a radiation phantom, rather than actual patient computed tomography (CT) data. The radiation phantom may be implemented in the form of any one of a wide variety of conventional radiation phantoms, including water-based phantoms and the like. The composition and geometry data preferably describes the impact of the target volume 12 on the transmission of treatment radiation beams 14 so that simulated portal images 16 may be computed.

In some intensity modulated radiation therapy (IMRT) embodiments, a digitally reconstructed portal image 16 may be computed as follows. Weights are assigned to the IMRT beams corresponding to a selected IMRT field based on the dose delivered for each segment in the IMRT field. Hence, a higher dose prescribed for a particular segment in a particular IMRT field results in a higher weight assigned for the beam and a higher CT value for the voxels in the target volume in the beam path. The composition and geometry data for the target volume are used to simulate the transmission impact of the various attenuation and scatter factors associated with the beam for a particular energy. A projection is created for each beam at, for example, an isometric plane or a beam view acquisition plane based upon a selected simulation modeling process (e.g., Monte Carlo modeling process). All of the projections for all of the beams of an IMRT field then are summed to obtain a composite digitally reconstructed portal image (DRIP) IMRT map. In some embodiments, a scatter model may be incorporated into the digitally reconstructed portal image generation process.

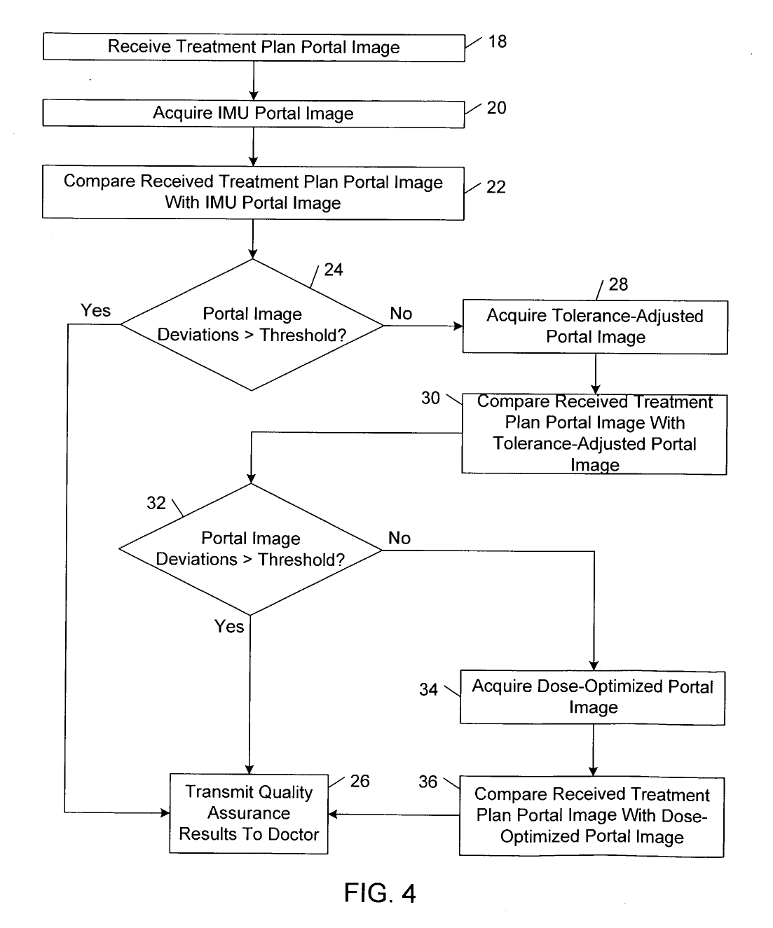

Referring to FIGS. 1 and 4, after the physicist receives the selected treatment plan from the doctor (step 18; FIG. 4), the physicist computes one or more independent monitor unit (IMU) DRPIs (step 20; FIG. 4) by using an independent monitor unit algorithm, different from the algorithms used by the treatment planning system. The actual QA portal images may be acquired by irradiating a target volume (e.g., a radiation phantom) having a known composition and geometry with radiation in accordance with a quality assurance plan that corresponds to the selected radiation therapy plan. The physicist compares the IMU DRPIs to the corresponding acquired QA portal images to independently verify the monitor unit calculations made by the dosimetrist (step 22; FIG. 4). If the deviations between the corresponding DRPIs exceed a threshold (step 24; FIG. 4), the physicist transmits the quality assurance results to the doctor, who may consider revising the radiation therapy plan (step 26; FIG. 4). Otherwise the physicist acquires one or more tolerance-adjusted DRPIs by adjusting the selected radiation therapy plan in accordance with known tolerances and performance metrics that are specific to the radiation therapy system that will be used to treat the patient and irradiating the target volume in accordance with the tolerance-adjusted radiation plan (step 28; FIG. 4). The physicist then compares the tolerance-adjusted DRPIs to the corresponding acquired QA portal images (step 30). If the deviations between the corresponding DRPIs exceed a threshold (step 32; FIG. 4), the physicist may decide to have the linear accelerator serviced and recalibrated (step 26; FIG. 4). Otherwise the physicist acquires one or more dose-optimized DRPIs by adjusting the selected radiation therapy plan in accordance with known dose optimization techniques and irradiating the target volume in accordance with the dose-adjusted radiation plan (step 34; FIG. 4).

The physicist compares the dose-optimized DRPIs with the corresponding acquired QA portal images (step 36; FIG. 4). The physicist then transmits the quality assurance results to the doctor for review (step 26; FIG. 4).

Referring to FIG. 1, after the doctor receives the quality assurance results, the doctor may decide to go forward with the selected plan or the doctor may select a different radiation therapy plan and repeat the quality assurance process.

After the doctor has selected a plan the meets the clinical objectives and meets the quality assurance standards, the doctor sends a final radiation therapy treatment plan (Final RT Plan) to a radiation therapy technician who will irradiate the patient using a medical radiotherapy device.

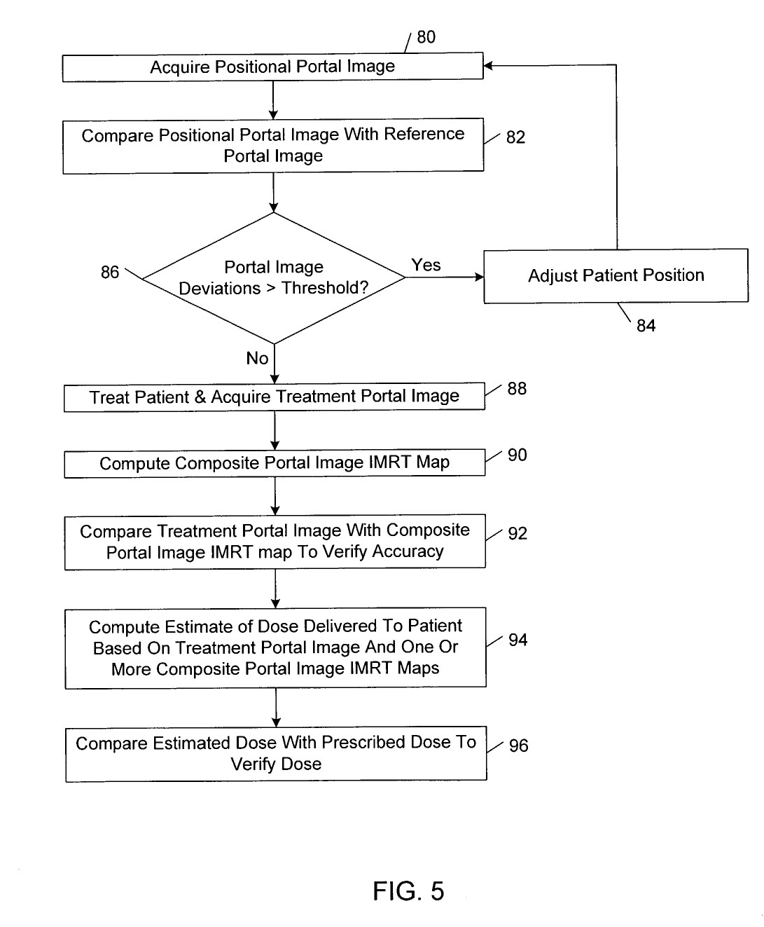

Referring to FIGS. 1 and 5, when the patient first arrives for treatment, the radiation therapy technician prepares the patient by immobilizing the patient on a support table of the radiotherapy device in rough alignment with the beam source. Next, the radiation therapy technician acquires one or more positional portal images of the patient (step 82; FIG. 5). The positional portal images may be obtained from high-energy (e.g., MeV) radiation treatment beams of very short duration. The portal images may be acquired by a conventional digital electronic portal imaging device or a conventional film-based x-ray imaging device. In x-ray film based embodiments, the acquires x-ray images may be scanned by a scanning device to obtain corresponding digital portal images. The radiation therapy technician then compares the positional portal images with one or more corresponding reference portal images (step 84; FIG. 5). In some embodiments, the reference portal images correspond to the treatment plan DRPIs that were generated by the physicist or the dosimetrist. The portal images are acquired using the same linear accelerator, multi-leaf collimator and table settings that were used to generate the reference DRPIs. The patient position is adjusted (step 84; FIG. 5) until the deviations between the acquired portal images and the corresponding reference portal images are below a threshold (step 86; FIG. 5).

After the patient has been positioned accurately (steps 80-86; FIG. 5), the radiation therapy technician treats the patient and acquires a portal image during the treatment (step 88; FIG. 5). During treatment, a prescribed radiation intensity profile typically is delivered to the patient at the dose tolerance limit once per day until the cumulative dose delivered to the tumor reaches the prescribed, optimal curative dose.

After the patient has been treated, the radiation therapy technician transmits the acquired treatment composite portal image to the doctor for verifying that the tumor was irradiated accurately and at the proper dose level. In IMRT embodiments, the doctor computes a composite portal image IMRT map based upon the IMRT treatment plan and patient CT data (step 90; FIG. 5). The composite portal image IMRT map may be computed by computing a projection for each beam of each IMRT field weighted based on beam doses respectively assigned by the treatment plan. Projections may be computed by determining the transmission impact of patient volumes through which beams of the IMRT field pass. The computed projections may correspond to an isocentric plane or a beam view acquisition plane. The composite portal image IMRT map is then computed by summing the projections for the beams of the IMRT fields.

Next, the radiation treatment may be evaluated based at least in part upon the computed composite portal image IMRT map and the acquired treatment portal image. For example, the treatment portal image may be compared with the computed composite portal image IMRT map to verify that the tumor was irradiated accurately (step 92; FIG. 5). In addition, the doctor may compute an estimate of the dose that was delivered to the patient based on the treatment portal image and one or more computed composite portal image IMRT maps (step 94; FIG. 5). The doctor then may compare the computed dosage level with the prescribed dosage level to verify that the tumor was irradiated at the correct dosage level (step 96; FIG. 5). In one embodiment, an estimate of the dose delivered to the tumor may be computed by computing a difference map based on subtraction between the treatment portal image and the composite portal image IMRT map. If the difference variations in the difference map are below a selected threshold, the treatment dose level may be assumed to correspond to the dose prescribed by the treatment plan. Otherwise, the difference map is back-projected through a CT model of the patient to a beam source corresponding to the IMRT fields. The back-projection process incorporates the transmission impact of patient volumes through which beams of the IMRT fields pass. The back-projected difference map then is applied to the original treatment plan to obtain a modified treatment plan. A second composite portal image IMRT map is computed based on the modified treatment plan and patient CT data. A second difference map is computed based on subtraction between the treatment portal image and the second composite portal image IMRT map. If the difference variations in the second difference map are below the selected threshold, the treatment dose level may be assumed to correspond to the modified treatment plan. Otherwise, the process is repeated until a difference map having difference variation below the selected threshold is obtained.

Other embodiments are within the scope of the claims. For example,

The systems and methods described herein are not limited to any particular hardware or software configuration, but rather they may be implemented in any computing or processing environment, including in digital electronic circuitry or in computer hardware, firmware, or software. In general, the systems may be implemented, in part, in a computer process product tangibly embodied in a machine-readable storage device for execution by a computer processor. In some embodiments, these systems preferably are implemented in a high level procedural or object oriented processing language; however, the algorithms may be implemented in assembly or machine language, if desired. In any case, the processing language may be a compiled or interpreted language. The methods described herein may be performed by a computer processor executing instructions organized, for example, into process modules to carry out these methods by operating on input data and generating output.

What is claimed is:

1. A method of planning a radiation therapy treatment, comprising:
computing a proposed treatment plan portal image based upon a proposed treatment plan;
acquiring a quality assurance portal image by irradiating a target volume having a known composition and geometry with radiation in accordance with a quality assurance treatment plan corresponding to the proposed treatment plan; and
evaluating the proposed treatment plan based on a comparison of the proposed treatment plan portal image and the quality assurance portal image.

2. The method of claim 1, wherein the target volume is a quality assurance radiation phantom.

3. The method of claim 1, wherein the proposed treatment plan is an intensity modulated radiation therapy plan.

4. The method of claim 1, wherein the quality assurance treatment plan corresponds to a tolerance-adjusted version of the proposed treatment plan.

5. The method of claim 1, wherein the quality assurance treatment plan corresponds to a dose-optimized version of the proposed treatment plan.

6. A method of verifying radiation therapy treatment, comprising:
computing a composite portal image IMRT map based on an intensity modulated radiation therapy (IMRT) treatment plan and computed tomography (CT) data for a patient; and
evaluating radiation delivered to the patient based at least in part on the composite portal image IMRT map and a treatment portal image acquired during delivery of a target dose of radiation to a target volume of the patient in accordance with the IMRT treatment plan.

7. The method of claim 6, wherein computing the composite portal image IMRT map comprises computing a projection for each beam of the IMRT field weighted based on beam doses respectively assigned by the treatment plan.

8. The method of claim 7, wherein computing the projection comprises determining transmission impact of patient volumes through which beams of the IMRT field pass.

9. The method of claim 7, wherein the computed projection corresponds to an isocentric plane or a beam view acquisition plane.

10. The method of claim 7, wherein computing the composite portal image IMRT map further comprises summing projections computed for the beams of the IMRT field.

11. The method of claim 6, wherein evaluating radiation delivered to the patient comprises determining correspondence between the target volume of the patient and the delivered radiation to verify irradiation accuracy.

12. The method of claim 6, wherein evaluating radiation delivered to the patient comprises computing an estimate of radiation dose delivered to the target volume of the patient based upon the treatment portal image and the composite portal image IMRT map.

13. The method of claim 12, wherein computing the radiation dose estimate comprises computing a difference map based on subtraction between the treatment portal image and the composite portal image IMRT map.

14. The method of claim 13, wherein computing the radiation dose estimate further comprises back projecting the difference map through a model of the patient to a beam source corresponding to the IMRT field.

15. The method of claim 14, wherein back projecting the difference map comprises determining transmission impact of patient volumes through which beams of the IMRT field respectively pass.

16. The method of claim 14, wherein computing the radiation dose estimate further comprises computing a modified treatment plan by applying the back-projected difference map to the IMRT treatment plan.

17. The method of claim 16, wherein computing the radiation dose estimate further comprises computing a modified treatment plan composite portal image IMRT map based on the patient CT data and the modified treatment plan.

18. The method of claim 17, wherein computing the radiation dose estimate further comprises computing a second difference map based on subtraction between the modified treatment plan composite portal image IMRT map and the treatment portal image.

19. The method of claim 18, wherein computing the radiation dose estimate further comprises identifying a dose prescribed by the modified treatment plan as the radiation dose estimate when the difference map indicates that the treatment portal image and the composite portal image IMRT map are substantially similar.

20. A method of verifying patient position in a radiation therapy treatment, comprising:
(a) computing a reference portal image based on a prescribed radiation beam and computed tomography (CT) data for a patient;
(b) acquiring a treatment portal image for the radiation beam;
(c) adjusting patient position based on a comparison of the reference portal image with the acquired portal image; and
(d) repeating steps (a)-(c) until the reference portal image and the acquired portal image match substantially.

21. A method of computing a dose delivered to a target during radiation treatment, comprising:
computing a projection for each beam of an IMRT field of a treatment plan based at least in part on transmission impact of patient volumes through which beams of the IMRT field respectively pass;
summing the computed beam projections to obtain a reconstructed composite portal image IMRT map; and
computing the dose and variations in dose delivered to the target by comparing the reconstructed composite portal image to a composite portal image acquired during radiation treatment.
